(19) United States
(12) Patent Application Publication
(10) Pub. No.: US 2005/0245874 A1
(43) Pub. Date: Nov. 3, 2005

(54) Device for locoregional anesthesia and method for making the cannula of said device

(75) Inventors: Jean-Luc Carrez, Ecouen (FR); Jean-Louis Coussegal, Beauchamp (FR); Valery Dalle, Gouvieux (FR)

Correspondence Address: LEVINE & MANDELBAUM, 444 MADISON AVENUE, 35TH FLOOR, NEW YORK, NY, 10022, US

(73) Assignee: Vygon, Ecouen (FR)

(21) Appl. No.: 10/519,926

(22) PCT Filed: Nov. 18, 2003
(86) PCT No.: PCT/FR03/03410

§ 371 Date: Dec. 30, 2004

(30) Foreign Application Priority Data
Nov. 20, 2002 (FR) 02/14527

(51) Int. Cl. A61M 5/178

(52) U.S. Cl. 604/160; 604/164.05

(57) ABSTRACT

The invention concerns a device for locoregional anesthesia, comprising an electrically stimulated puncture needle (3) provided with a fixed hub (4), a cannula (12) and a catheter (15), said cannula consisting of a tube (1) and a fixed hub (2) adapted to be mounted on the hub (4) of the needle and the catheter being adapted to slide inside the cannula. The invention is characterized in that the cannula has from end to end a longitudinal slot (6, 7) enabling the cannula to be laterally separated from the catheter through said slot. The invention is applicable to anesthesia of a limb or a specific part of the body.

This invention relates to a locoregional anaesthesia device and a canula for this device.

Locoregional anaesthesia consists of anaesthetizing a limb or a defined part of the body. The now traditional technique for doing this consists of inserting an electrically stimulable punction needle as close as possible to the bundle of nerves concerned. This positioning is made possible when very close to the nerves concerned by using a stimulation by means of a small electrical current that is sufficient to trigger the muscular response. At this moment, an anaesthetizing block is injected through the needle and the needle is withdrawn.

It is not desirable to inject a large block, so when reinjection is necessary during the operation (if it is longer than expected) or for post-operational purposes (pain/re-education), a catheter is inserted to enable this reinjection.

One known technique of inserting this catheter consists of using a stimulation needle on which a canula is temporarily fixed that frees the bevelled distal end of the needle;

After needling and stimulation, the needle is withdrawn, leaving the canula in place that is used to insert and place the catheter that will be used for re-injection of the anaesthetic, after which the canula is removed.

One purpose of the invention is to facilitate placement and removal of the canula and to avoid risks of moving the re-injection catheter during removal of the canula.

This is achieved according to the invention by using a device for locoregional anaesthesia comprising a needle, a canula and a catheter, the canula being composed of a short shaft fitted with a fixed hub that can be mounted on the base of the needle and the catheter that can be inserted in the canula, the device being characterised by the fact that the canula has a longitudinal slit from end to end by which the canula can be separated laterally from the catheter through this slit.

The completely slitted canula is a new medical accessory for placement of a catheter.

One method of manufacturing this canula consists of making a slitted hub with a wide slit, tapering one end of an unslitted shaft on a conical mandrel, applying and gluing this tapered end in the slit in the hub (for example, using a glue that can be polymerised under U.V.

Advantageously, the hub and the shaft of the canula are made using different synthetic resins such that the hub is relatively rigid and the shaft is relatively flexible.

The slit in the hub is wide enough to allow the catheter to pass through the slit, while the slit in the shaft may be narrower than the catheter but the lips of the slit may move apart to enable the catheter to be forced fitted through the slit.

In preferred embodiments, the device according to the invention has one or more of the following characteristics:
the shaft has a tapered proximal end (8) glued into the slit in the hub of the canula;
the hub of the canula and the hub of the needle are provided with means (10-13) that cooperate to lock the hub of the canula onto the hub of the needle and make release possible by relative rotation of the two hubs;
the said cooperating locking means are side teeth (10, 11) on the back of the hub of the canula and retaining tabs (12, 13) at the front of the hub of the needle;
the catheter of the device is provided with a fixed hub;

We will now describe an example embodiment of a canula with a base complying with the invention, with reference to the figures in the appended drawing wherein:

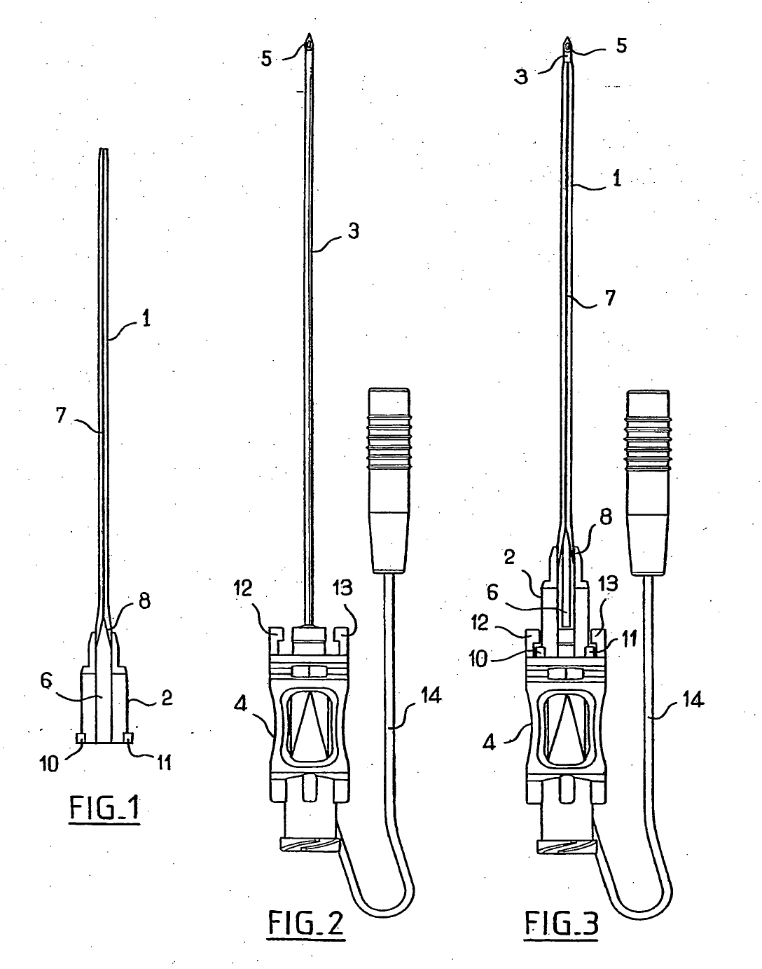

FIG. 1 is a longitudinal diagram of the canula with its hub;

FIG. 2 is a longitudinal diagram of a neurostimulation needle;

FIG. 3 is a diagram of the canula mounted on the needle;

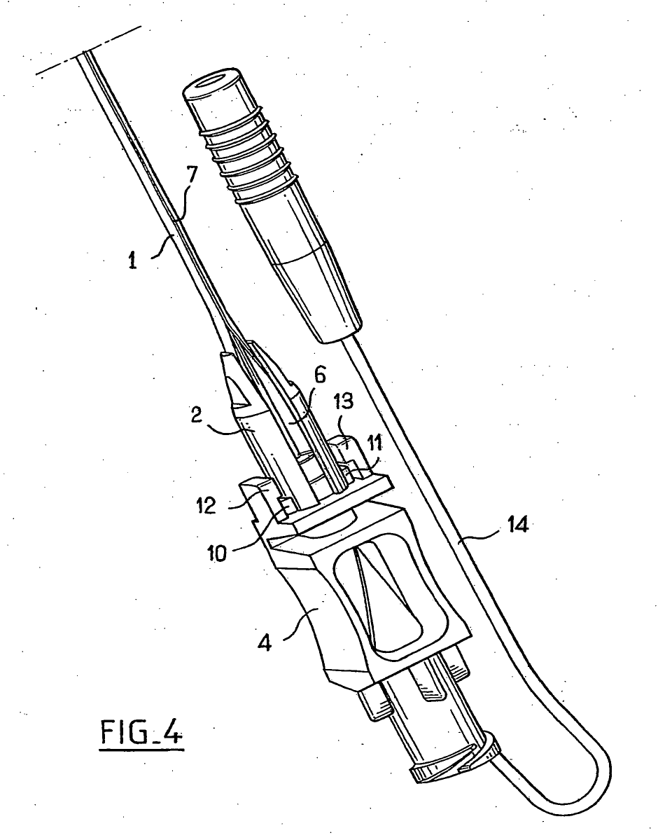

FIG. 4 is an enlarged view of the hub of the canula locked on the hub of the needle;

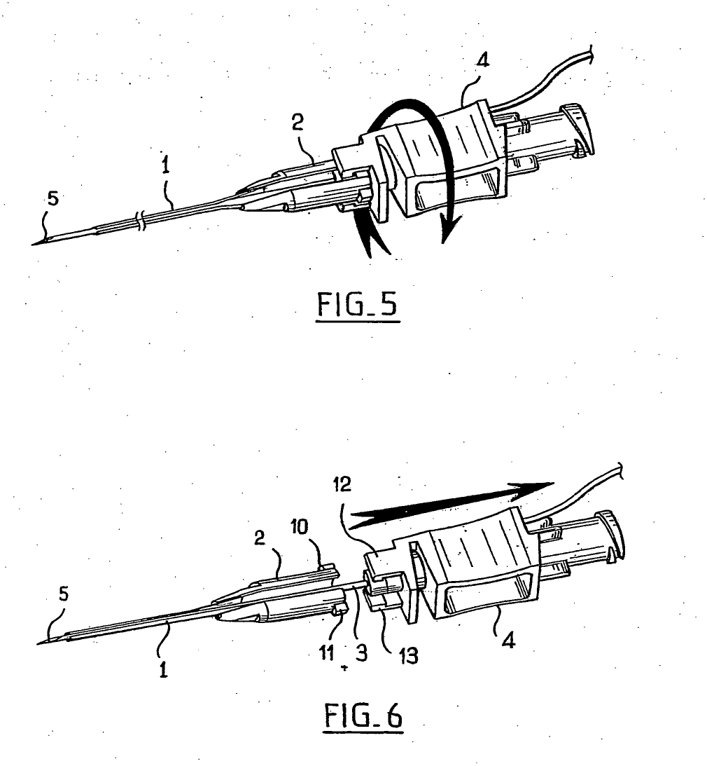

FIG. 5 is a view of the hub of the canula on the hub of the needle after the unlocking rotation;

FIG. 6 is a view of the hub of the needle during extraction of the needle; and

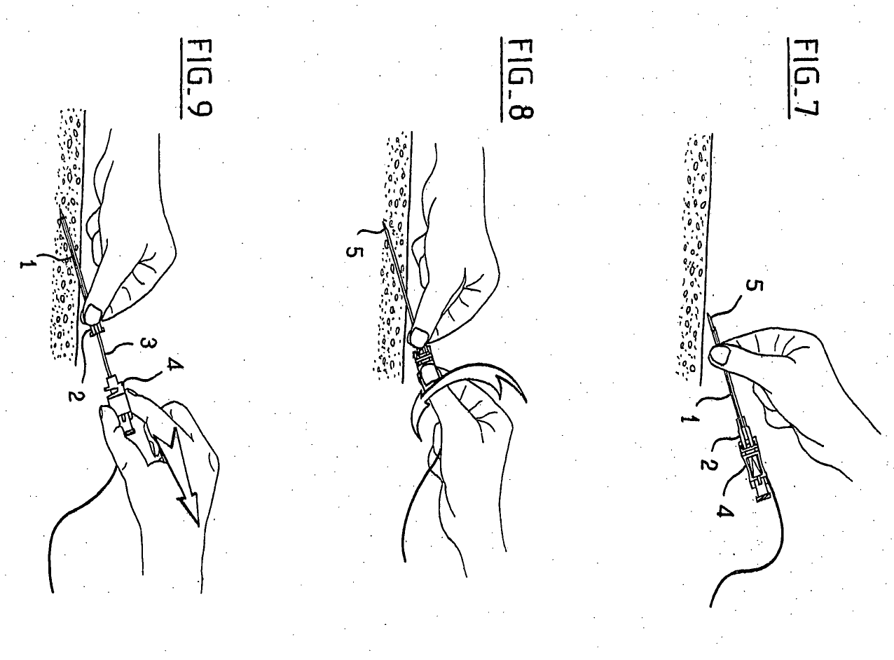

FIGS. 7 to 12 show successive views during a manipulation to put a catheter into place using a canula with a hub according to the invention.

The canula according to the invention (FIG. 1) is composed of a shaft (1) and a hub (2) fixed to the tube, and will be installed on a needle (3) (FIG. 2) such that the hub (2) is locked (and can be unlocked on request) on the hub (4) of the needle, while the bevelled tip (5) of the needle remains visible (FIG. 3).

The hub (2) of the canula has a wide longitudinal slit (6) and the shaft (1) of the canula has a narrow slit (7) aligned with the slit in the hub. FIG. 3 shows the tapered proximal end (8) of the shaft fixed in the end of the slit (6) in the hub (2), such that the internal face of the shaft is perfectly contiguous with the internal face of the hub, so that the transition between the hub and the shaft will not cause a displacement of the catheter when the canula is being removed.

The hub (2) of the tube is provided with two teeth (10, 11) at the back and the hub (4) of the needle has two L-shaped tabs (12, 13) at the front that are capable of retaining the two teeth (FIG. 3), while they can be released by rotating the hub of the shaft.

The hub of the needle is designed such that the needle can be electrically connected to a current source using a conductor (14), in a manner known in itself.

The mechanical attachment system of the canula creates a fixity with the needle that makes it possible to push while maintaining the assembly very close to the needling point, which is better when large forces are necessary.

The canula can be released from the needle by making a 90° rotation.

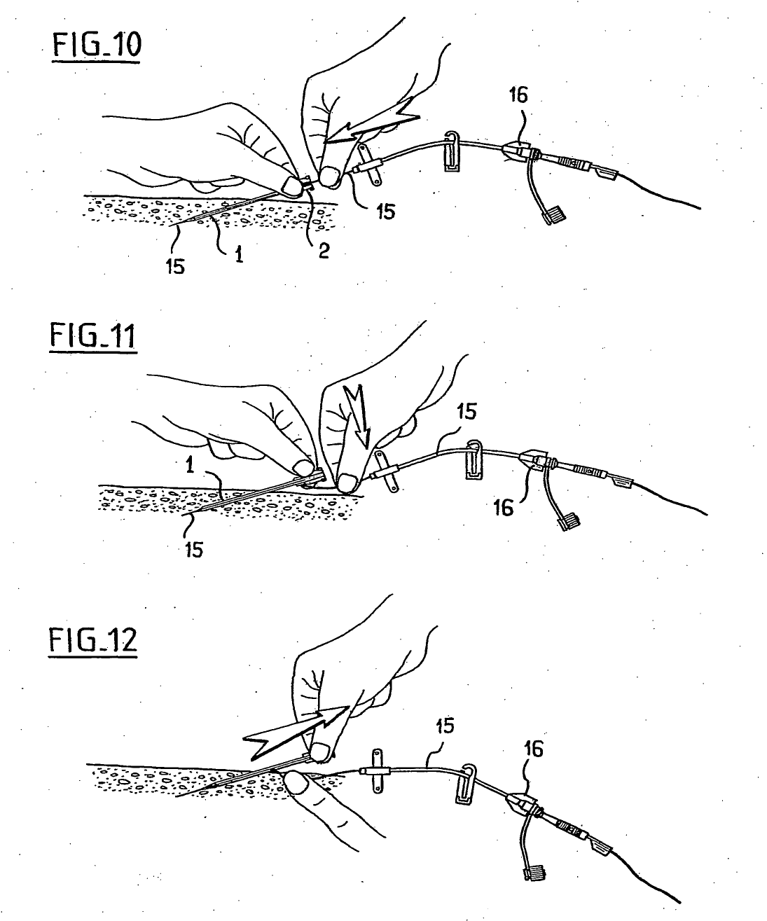

The device for placement of a catheter (15) is used as follows:
the device is extracted from a sterile pack containing the needle, the canula and the catheter;
the skin is needled using the needle on which the canula is mounted (FIG. 6);
the (needle+canula) assembly is held close to the needling point and the nerve is identified by electrical stimulation;
when the nervous stimulation has been made, the needle is separated from the canula by pivoting and extracting the needle, the canula being held in place manually (FIGS. 7, 8);
the catheter is inserted into the canula as far as a mark made on the catheter (FIG. 9);
the canula is gradually removed by moving the canula aside laterally and sliding the canula along the catheter, the catheter being held in place manually to avoid tension being applied to the catheter (FIGS. 10, 11).

The inserted catheter may be equipped with a metallic mandrel for electrical stimulation.

Advantageously, a long catheter (15) will be used fitted with a fixed proximal hub (16) or a central hub with a flexible extension fitted with a proximal hub.

The invention is not limited to the embodiment that has been described.

1. Device for locoregional anaesthesia comprising a punction and electrical stimulation needle (3) fitted with a fixed hub (4), a canula (1, 2) and a catheter (15), this canula being composed of a shaft (1) and a fixed hub (2) that can be mounted on the hub (4) of the needle, and the catheter being able to slide in the canula, characterised in that the canula has a longitudinal slit (6, 7) from end to end by which the canula can be separated laterally from the catheter through this slit.

2. Device according to claim 1, in which the hub (2) and the shaft (1) of the canula are made using different synthetic resins.

3. Device according to claim 1 or 2, in which the shaft (1) of the canula has a tapered proximal end (8) glued into the slit (6) in the hub of the canula.

4. Device according to any of claims 1 to 3, in which the hub of the canula and the hub of the needle are provided with means (10-13) cooperating to lock the hub of the canula onto the hub of the needle and make release possible by relative rotation of the two hubs.

5. Device according to claim 4, in which the said cooperating locking means are side teeth (10, 11) on the back of the hub of the canula and retaining tabs (12, 13) at the front of the hub of the needle.

6. Device according to any of claims 1 to 5, in which the slit (7) in the shaft of the canula is narrower than the catheter but may move apart to enable the catheter to be forced fitted outside the slit (6), whereas the slit (6) in the hub of the canula is wide enough to allow the catheter to pass through.

7. Device according to any of claims 1 to 6, the catheter (15) of which is fitted with a fixed hub (16).

8. Device according to any of claims 1 to 7, and which includes a pack containing the needle, the canula and the catheter.

9. Canula for a locoregional anaesthesia device according to one of claims 1 to 8, this canula being composed of a shaft (1) and a fixed hub (2), characterised in that the canula has a longitudinal slit (6, 7) from end to end.

10. Method of manufacturing a canula according to claim 9, in which a slitted hub is made, one end of an unslitted shaft is tapered, this shaft end is inserted into the slit in the hub until it stops in contact with a limit stop and is fixed in this slit by gluing, and the shaft is slitted longitudinally in line with the slit in the hub.
